(19) United States
(12) Patent Application Publication
(10) Pub. No.: US 2026/0022332 A1
(43) Pub. Date: Jan. 22, 2026

(54) CHIMERIC SURFACES AND USES THEREOF

(71) Applicant: THE PENN STATE RESEARCH FOUNDATION, University Park, PA (US)

(72) Inventors: Pak Kin Wong, University Park, PA (US); Ninghao Zhu, University Park, PA (US); Mona Ahmed, University Park, PA (US); Tak Sing Wong, University Park, PA (US)

(21) Appl. No.: 19/116,716

(22) PCT Filed: Oct. 3, 2023
(86) PCT No.: PCT/US2023/075886

§ 371 Date: Mar. 28, 2025

Related U.S. Application Data
(60) Provisional application No. 63/378,179, filed on Oct. 3, 2022.

(51) Int. Cl. C12N 5/00; C12M 1/00; C12N 5/09; G01N 33/50

(52) U.S. Cl. CPC C12N 5/0068; C12M 23/20; C12N 5/0693; G01N 33/5091; C12N 2503/00; C12N 2513/00; C12N 2533/30

(57) ABSTRACT

The present disclosure provides chimeric surfaces. The chimeric surfaces may be thin films. In various embodiments, the thin films may be disposed on a substrate. Articles of manufacture may have one or more chimeric surfaces. Also disclosed are methods of making organoids from cancer cells and methods of determining whether a subject has cancer. The thin films may be formed from one or more siloxanes and one or more lubricants.

### CROSS REFERENCE TO RELATED APPLICATIONS

This application claims the benefit of U.S. Provisional Application No. 63/378,179, filed on Oct. 3, 2022, the disclosure of which is incorporated herein by reference.

### BACKGROUND OF THE DISCLOSURE

Cancer is one of the major causes of death worldwide. For example, bladder cancer is the sixth most common cancer in the United States, accounting for an estimated over 80,000 new cases in 2022. In spite of the advances of current treatments, the recurrence rate of bladder cancer is 50% to 70% within five years. Hence, it is vital to discover new diagnostic and therapeutic targets to improve the current treatments and monitor the patients after treatments. To date, several lncRNAs have been identified to be aberrantly expressed in bladder cancer. High expression of MALAT1 can promote the bladder cancer migration by triggering EMT, and the clinical reports indicated more bladder tumor metastasis with high level of MALAT1 expression. Similarly, lncRNA UCA1 (urothelial carcinoma associated 1) was reported to promote bladder cancer invasion and EMT, with UCA1 targeting at miR-143/HMGB 1 pathway. These studies reveal the important role of lncRNAs in bladder cancer invasion, providing potential new therapeutic targets to combat bladder cancer. However, given the reported contradictory functions of MALAT1 in cancers, a comprehensive study of MALAT1 in bladder cancer invasion is warranted for validating the therapeutic potential.

Since leader cells represent only a small subset of cancer cells, live single cell biosensors with a high spatiotemporal resolution are required for investigating the function of lncRNA. However, existing techniques, such as RNA sequencing and RNA fluorescence in situ hybridization (FISH) that lyse or fix the samples, fail to reveal the spatial and temporal dynamics of RNAs in cancer cells during collective cancer invasion.

Bladder cancer prognosis, involving staging and grading, relies on histopathology. Tumor-Node-Metastasis (TNM) system classifies the bladder cancer as different stages (Stage I to Stage IV) according to the penetration depth into the surrounding tissues and different grades (high grade and low grade) according to the aggressiveness of the tumor cells.

Accurate staging and grading predict the progression of bladder cancer and the effectiveness of the treatment, assisting the physicians to recommend appropriate treatments and to evaluate the recovery. The gold standard of the bladder cancer prognosis includes urine tests, cystoscopy, imaging tests (e.g., CT scan, MRI and PET scan), and histology from biopsy or transurethral resection of bladder tumor (TURBT). The urine tests for cancer biomarkers provide rapid and noninvasive testing. However, due to the complexity of bladder cancer metastasis, limited number of biomarkers cannot comprehensively predict the progression of bladder cancer, and it fails to consider the tumor invasion into the surrounding tissue. Imaging-based methods can reflect the morphology of the cancer cells and the penetration of tumor, but they rely on the experience of physicians and fail to analyze the gene expression. Therefore, there is a critical need for a combined genotypic and phenotypic assay for bladder cancer prognosis, considering both the vital biomarkers and the morphology of tumor cell invasion.

### SUMMARY OF THE DISCLOSURE

The present disclosure provides chimeric surfaces. The chimeric surfaces may be thin films. In various embodiments, the thin films may be disposed on a substrate. Articles of manufacture may comprise one or more chimeric surfaces. Also disclosed are methods of making organoids and methods of determining whether a subject has cancer.

In an aspect, the present disclosure provides films, which may be thin films. The thin films may comprise one or more siloxanes and one or more lubricants.

In an aspect, the present disclosure provides articles of manufacture. In various examples, the articles of manufacture are used in cell culture or are cell culture devices. Non-limiting examples of such articles include cell culture plates, multi-well plates, test tubes, flasks, or larger vessels such bioreactors.

In an aspect, the present disclosure provides methods of making a film of the present disclosure. The films may be made directly onto a desired surface.

In an example, the film is produced by contacting a siloxane film with a lubricant. The siloxane film may be deposited (e.g., placed or formed) on a desired surface and heated (e.g., heated to 70° C. for 2 hours) to form a flat layer of siloxane. The lubricant may then be contacted with the siloxane and heated (e.g., incubated at 55° C. overnight). Excess lubricant may then be removed (e.g., aspirated). Following removal of excess lubricant, at least a portion of the lubricant remains in contact (e.g., bound) to the siloxane. The film may then be sterilized. For example, the sterilization may be via irradiation with UV light.

In an aspect, the present disclosure provides methods of initiating self-assembly cells to form organoids. The cells may be suspended in a cell culture medium and contacted with the film.

In an example, a method of the present disclosure comprises contacting one or

more cells with a film of the present disclosure. After contacting, the film, self-assembly into organoids is initiated. After a period 1, 2, 3, 4, 5, 6, or 7 days, an organoid comprising the cells is formed. This organoid can be further disseminated.

In various examples, a method of the present disclosure may be used to determine if a subject has cancer (e.g., invasive cancer). The method may comprise culturing one or more cells isolated from the subject in the presence of a film of the present disclosure, wherein the formation of an organoid and dissemination of cancer cells from the organoid in contact with the film is indicative of the subject having cancer (e.g., invasive cancer).

### BRIEF DESCRIPTION OF THE FIGURES

For a fuller understanding of the nature and objects of the disclosure, reference should be made to the following detailed description taken in conjunction with the accompanying figures.

FIG. 1 shows schematics of the chimeric invasion assay (CIA).

FIG. 2 shows MALAT1 expression in 3D human tumor organoids derived from bladder cancer patients of different cancer stages cultured on CIA on day 0 and day 3. Images are representative of three experiments. Scale bars, 300 μm.

FIG. 3 shows human tumor organoids self-assembly on PDMS surface (a) and chimeric surface (b). Images are representative of three experiments. Scale bars, 300 μm.

FIG. 4 shows actin filament staining of human tumor organoids self-assembled on PDMS surface (a) and chimeric surface (b). Images are representative of three experiments. Scale bars, 50 μm.

FIG. 5 shows quantification of human tumor organoids on CIA. a, Quantification of ellipticity (oblate) of human tumor organoids on CIA. b, Quantification of MALAT1 expression level of human tumor organoids on CIA. One-way ANOVA followed by Tukey's post hoc test was used to compare the ellipticity and MALAT1 expression level from spheroids (n=221, 216, 365, and 437 for DT2334, DT2101, DT2153 and DT2296, NS, not significant, * p<0.05, **, p<0.01, ***, p<0.001, ****, p<0.0001).

FIG. 6 shows X-ray photoelectron spectroscopy (XPS) characterization of different surfaces. CM: cell culture media. Data were acquired from three experiments.

FIG. 7 shows Fourier-transform infrared spectroscopy (FTIR) characterization of different surfaces. a, Absorbance curve of FTIR of different surfaces. b, Normalized protein retention on different surfaces. Data were acquired from three experiments.

FIG. 8 shows self-assembly of patient-derived cells (SH2) on CIA on day 3 with MALAT1 labeling. The image is representative of six repeats. Scale bar, 300 μm.

FIG. 9 shows tumor organoids (formed with sub-cultured TURBT cells, SH1 and SH2) with MALAT1 nanobiosensor cultured on 3D invasion assay (a) and chimeric assay (b). Scale bars, 200 μm. c, Quantification of ellipticity (oblate) of tumor organoids on CIA. d, Quantification of MALAT1 expression level of tumor organoids on CIA. Student's t-test was used to compare the ellipticity and MALAT1 expression level from spheroids (n=1183 and 1068 for SHI and SH2, **, p<0.01, ****, p<0.0001).

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Throughout this application, the use of the singular form encompasses the plural form and vice versa. For example, “a”, or “an” also includes a plurality of the referenced items, unless otherwise indicated.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

The present disclosure provides chimeric surfaces. The chimeric surfaces may be thin films. In various embodiments, the thin films may be disposed on a substrate. Articles of manufacture may comprise one or more chimeric surfaces. Also disclosed are methods of making organoids and methods of determining whether a subject has cancer.

In an aspect, the present disclosure provides films, which may be thin films. The thin films may comprise one or more siloxanes and one or more lubricants.

The films may comprise various siloxanes or groups formed therefrom. The films may be referred to as layers. For example, the siloxane may be a polymeric siloxane. Examples of siloxanes include, but are not limited to polydimethylsiloxanes (PDMS), polymethylhydrosiloxanes (PMHS), octamethylcyclotetrasiloxanes, and the like, and combinations thereof. In various examples, other similar elastomers may be used. In various examples, the siloxane may comprise one or more reactive groups, which may be cross-linkable groups, such as, for example, one or more OH groups, one or more trimethylsilyl groups, and/or one or more vinyl groups. In various examples, the siloxane is PDMS. Examples of suitable PDMS include, but are not limited to, SYLGARD 184®, SYLGARD 527R, and the like, and combinations thereof. The siloxane may be cross-linked (e.g., fully cross-linked, where all cross-linking groups have been reacted) or partially cross-linked (e.g., where only a portion of the cross-linking groups are reacted). For example, the PDMS may be a cross-linked PDMS (e.g., partially cross-linked or fully cross-linked). In various examples, PDMS for use in the present disclosure is formed from a prepolymer and a curing agent.

PDMS (e.g., cross-linked PDMS) may have various ratios of its elastomeric components and cross-linking components. For example, concentration of the cross-linking component is 3.3 to 33% (i.e., 1:30 to 1:3), including all 0.1% values and ranges therebetween, relative to the total amount of elastomeric component and cross-linking component, where the total percentage does not exceed 100%. In various examples, the ratio of elastomeric components to cross-linking components is 10:1. The “elastomeric component” may be referred to as “elastomer” and the “cross-linking component” may be referred to as “cross-linker.”

The film may comprise various lubricants. The lubricant may be biocompatible. In various examples, the lubricant is a perfluoroalkyl ether (PFAE). In various examples, the PFAE has the following structure:

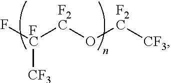

where n is 10 to 60, including all integer values and ranges therebetween. Examples of suitable lubricants include, but are not limited to, commercially available KRYTOX® lubricants (e.g., KRYTOX® 101, KRYTOX® 102, KRYTOX® 103, KRYTOX® 104, KRYTOX® 105, KRYTOX® 106, KRYTOX® 107, and the like, and combinations thereof). In various examples, the lubricant has a desirable viscosity. For example, the viscosity may be 7.8 to 450 cSt (7.8 to 450 mm²/s), including all 0.01 values and ranges therebetween.

In various examples, the film may comprise various amounts of lubricant. The amount of lubricant may cause the film to swell. For example, the substrate may comprise 100 nm to 10 mm lubricant, including all integer values and ranges therebetween. In various examples, the thickness ratio of lubricant to PDMS is 1:100 to 1:1, including all 0.1 ratio values and ranges therebetween, where the thickness ratio to lubricant to non-swollen PDMS.

The film may have various desirable features. For example, the film may be porous. The pores may have various dimensions. For example, the long linear dimension of each pore may be 0.2 nm to 20 μm. The porosity of the thin film may be uniform or non-uniform. For example, portions of the film may be porous and other portions are not porous; however, at least a portion of the film is porous. In various examples, at least a portion of the lubricant may be in the pores of the film.

In various examples, at least a portion of the lubricant is in contact with a surface of a siloxane. For example, a siloxane may be a siloxane layer or film and the lubricant may contact the siloxane layer or film such that at least a portion of a surface of the siloxane layer or film is contacted or was contacted with the lubricant. For example, there may be an excess of lubricant contacting a surface of the siloxane layer.

In various examples, the siloxane layer is coated with the lubricant.

In various examples, a siloxane film is impregnated with a lubricant.

Additionally, the film may have various thicknesses. For example, the film may be 2 μm to 2 cm in thickness, including all integer nanometer values and ranges therebetween. The thickness may be uniform or non-uniform. For example, portions of the film may be about 2 μm and other portions may be thicker or than 2 μm. The film may also have a desirable elasticity. For example, the film has a Young's modulus of 20 to 2500 kPa, including all 0.1 values and ranges therebetween.

In various embodiments, the film is used to coat all or a portion of a cell culture device, including, but not necessarily limited to cell culture plates, multi-well plates, test tubes, flasks, or larger vessels such bioreactors. In embodiments, the film is used on all or part of a cell culture device that comes into contact with cells.

In an aspect, the present disclosure provides articles of manufacture. In various examples, the articles of manufacture are used in cell culture or are cell culture devices. Non-limiting examples of such articles include cell culture plates, multi-well plates, test tubes, flasks, or larger vessels such bioreactors.

The article of the manufacture may have a plurality of surfaces. At least a portion of one or more surfaces may have the film of the present disclosure disposed thereon. The film may be coated or deposited on the surface or the film may be synthesized directly on a desired surface.

In an aspect, the present disclosure provides methods of making a film of the present disclosure. The films may be made directly onto a desired surface.

In an example, the film is produced by contacting a siloxane film with a lubricant. The siloxane film may be deposited (e.g., placed or formed) on a desired surface and heated (e.g., heated to 70° C. for 2 hours) to form a flat layer of siloxane. The lubricant may then be contacted with the siloxane and heated (e.g., incubated at 55° C. overnight). Excess lubricant may then be removed (e.g., aspirated). Following removal of excess lubricant, at least a portion of the lubricant remains in contact (e.g., bound) to the siloxane. The film may then be sterilized. For example, the sterilization may be via irradiation with UV light.

In an aspect, the present disclosure provides methods of initiating self-assembly cells to form organoids. The cells may be suspended in a cell culture medium and contacted with the film.

In an example, a method of the present disclosure comprises contacting one or more cells with a film of the present disclosure. After contacting, the film, self-assembly into organoids is initiated. After a period 1, 2, 3, 4, 5, 6, or 7 days, an organoid comprising the cells is formed. This organoid can be further disseminated.

In various examples of the method, the film is disposed on an article as described herein. Non-limiting examples of such articles include cell culture plates, multi-well plates, test tubes, flasks, or larger vessels such bioreactors.

Various cell culture mediums may be used in a method of the present disclosure. For example, the medium, such as Dulbecco's Modified Eagle's Medium (DMEM), may comprise different proteins, such as, for example, fetal bovine serum (FSB) at 0 to 20%, bovine serum albumin (BSA), or other proteins. The cell culture may comprise one or more of FSB or BSA. In various examples, the medium comprises cells with FSB. In various other examples, the medium comprises cells without FSB.

Various cells may be used in the method. For example, the cells may be mammalian cells and associated with cancer. For example, the cancer may be bladder cancer, thus the cells are bladder cancer cells. The cancerous cells may be at various cancer stages.

In embodiments, a film of this disclosure is in physical contact with a cell culture medium and/or cells. In embodiment a film of this disclosure is in contact with cells including, but not necessarily limited, to mammalian cells, such as human cells. In embodiments, the cells are present in a 3D culture, such as an organoid. In an embodiment, the organoid is a patient-derived organoid.

In embodiments, the cells are cancer cells. In a non-limiting embodiment the cancer cells are human cancer cells, including but not necessarily limited to bladder cancer cells. In embodiments, the film is used for tumor spheroid self-assembly and dissemination. In embodiments, the film is used in conjunction with an assay to determine cancer cell invasiveness. In an embodiment, the assay is an assay of the presence, absence, amount, or location of a polynucleotide within cells. In an embodiment the film is used to analyze lncRNA MALAT1 in the invading fronts of cancer cells.

In various examples, a method of the present disclosure may be used to determine if a subject has cancer (e.g., invasive cancer). The method may comprise culturing one or more cells isolated from the subject in the presence of a film of the present disclosure, wherein the formation of an organoid and dissemination of cancer cells from the organoid in contact with the film is indicative of the subject having cancer (e.g., invasive cancer).

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements provide various embodiments of the present disclosure.
Statement 1. A thin film comprising one or more siloxanes and one or more lubricants, wherein at least a portion of the one or more lubricants is contacting a surface of the one or more siloxanes.
Statement 2. A thin film according to Statement 1, wherein the one or more siloxanes are chosen from polydimethylsiloxanes (PDMS), polymethylhydrosiloxanes (PMHS), octamethylcyclotetrasiloxanes, and any combination thereof.
Statement 3. A thin film according to Statement 2, wherein the one or more siloxanes are PDMS.
Statement 4. A thin film according to Statements 2 or 3, wherein the PDMS comprises an elastomer component and a cross-linker component and cross-linker component has a concentration of 3.3 to 33% relative to the total percentage of elastomer and cross-linker.
Statement 5. A thin film according to any one of Statements 2 to 4, wherein the PDMS has ratio of 10:1 elastomer component to cross-linker component.
Statement 6. A thin film according to any one of the preceding Statements, wherein the one or more lubricants are perfluoroalkyl ethers (PFAEs).
Statement 7. A thin film according to any one of the preceding Statements, wherein the one or more lubricants have the following chemical structure:

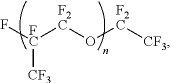

wherein n is 10 to 60.
Statement 8. A thin film according to any one of the preceding Statements, wherein the one or more lubricants have a viscosity of 7.8 cSt to 450 cSt, including all 0.01 cSt values and ranges therebetween.
Statement 9. A thin film according to any one of the preceding Statements, wherein the pores have a longest linear dimension of 0.2 nm to 20 μm, including all 0.01 nm values and ranges therebetween.
Statement 10. A thin film according to any one of the preceding Statements, wherein the thin film has a thickness of 2 μm to 2 cm, including all nm values and ranges therebetween.
Statement 11. A thin film according to any one of the preceding Statements, wherein the thin film has a Young's modulus of 20 to 2500 kPa, including all integer Pa values and ranges therebetween.
Statement 12. An article of manufacture having one or more surfaces, wherein a thin film according to any one of preceding Statements is disposed on at least a portion of at least one surface of the article of manufacture.
Statement 13. An article of manufacture according to Statement 12, wherein the article of manufacture is a cell culture device.
Statement 14. The article of manufacture according to claim 13, wherein the cell culture device is a cell culture plate, a multi-well plate, a test tube, a flask, or a bioreactor.
Statement 15. A method of producing an organoid, comprising contacting one or more cells with a thin film according to any one of Statements 1 to 11.
Statement 16. A method according to Statement 15, wherein the thin film is disposed on a surface of an article of manufacture according to any one of Statements 12 to 14.
Statement 17. A method according to Statement 16, wherein the article of manufacture is a cell culture device.
Statement 18. A method according to Statement 17, wherein the cell culture device is a cell culture plate, a multi-well plate, a test tube, a flask, or a bioreactor.
Statement 19. A method according to any one of Statements 15 to 18, wherein the one or more cells are mammalian cells.
Statement 20. A method according to Statement 19, wherein the mammalian cells are cancer cells.
Statement 21. A method according to Statement 20, wherein the cancer cells are bladder cancer cells.
Statement 22. A method for determining if a subject has cancer, comprising culturing one or more cells isolated from the subject in the presence of a thin film according to any one of Statements 1 to 11, wherein the formation of an organoid in contact with the thin film is indicative of the subject having cancer.
Statement 23. A method according to Statement 22, wherein the cancer is bladder cancer.

The following examples are presented to illustrate the present disclosure. They are not intended to be limiting in any matter.

### Example 1

This example provides a description of a surface of the present disclosure.

The assay described herein can be used predict bladder cancer's invasiveness. The current gold standard in clinical practice to determine the stage and grade of the bladder cancer includes urine tests, cystoscopy, imaging tests (e.g., CT scan, MRI and PET scan), and histology from biopsy or transurethral resection of bladder tumor (TURBT). To characterize the bladder tumor samples, genotypic analysis that studies the oncogene expression and phenotypic analysis that studies the morphology of the tumor are two directions. MALAT1 was previously identified as an important molecular biomarker in collective cancer invasion, and probing MALAT1 in human patient samples shows the potential of predicting the bladder cancer stage. This examples provides a 3D invasion assay used to analyze the invasiveness of the patient samples. Existing 3D invasion models includes embedded spheroid invasion assay and Boyden chamber assay. Nevertheless, these assays require intensive laboratory analysis and is not compatible with the clinical TURBT samples due to the lack of self-assembly capability. A chimeric invasion assay (CIA) is used herein to initiate tumor organoid self-assembly from patient-derived tumor cells. Combined with MALAT1 nanobiosensing, this assay was applied to bladder cancer prognosis.

### Fabrication of Chimeric Surface.

The chimeric surface was constructed by filling the porous structure of polydimethylsiloxane (PDMS) with biocompatible lubricants. The PDMS (Dow Corning, Midland, MI) was mixed with a 10:1 prepolymer to curing agent ratio. Fifty μL of PDMS was added to each well of 48 well plate and baked at 70° C. for two hours to form a flat layer of PDMS. Next, 100 μL of Krytox™ GPL 103 oil (Krytox, the Chemours Company, Wilmington, Delaware) was added to each well of 48 well plate on the PDMS and incubated at 55° C. overnight. The Krytox was then aspirated, and the chimeric surface was formed and ready to use after UV light sterilization.

### Tumor Cell Self-Assembly on Chimeric Invasion Assay.

Human bladder cancer dissociated tumor cells (stage II, lot number: DT02101 and DT02334; stage IIIB, lot number: DT02153; stage IV, lot number: DT02296) were purchased from BioIVT. Three patient samples (SH1, SH2 and SH3) were obtained from TURBT samples generously provided by Dr. Joseph C. Liao from Veterans Affairs Palo Alto Health Care System. After receiving the TURBT sample on the second day of the surgery, the tumor cells were obtained according to known methods. The tumor cells were transfected with MALAT1 FRET nanobiosensor for 24 hours. Afterwards, the tumor cells were seeded on the chimeric surface in 48 well plate with a density of 10,000 cells per well and incubated for 24 hours to form spheroids. The Invasion Matrix was then added to the spheroids and organoids. After gel formation for an hour, fresh culture media were added. The spheroids and organoids were then imaged at 0, 24, 48 and 72 hours.

### Tumor Spheroids Self-Assembly and Dissemination on CIA.

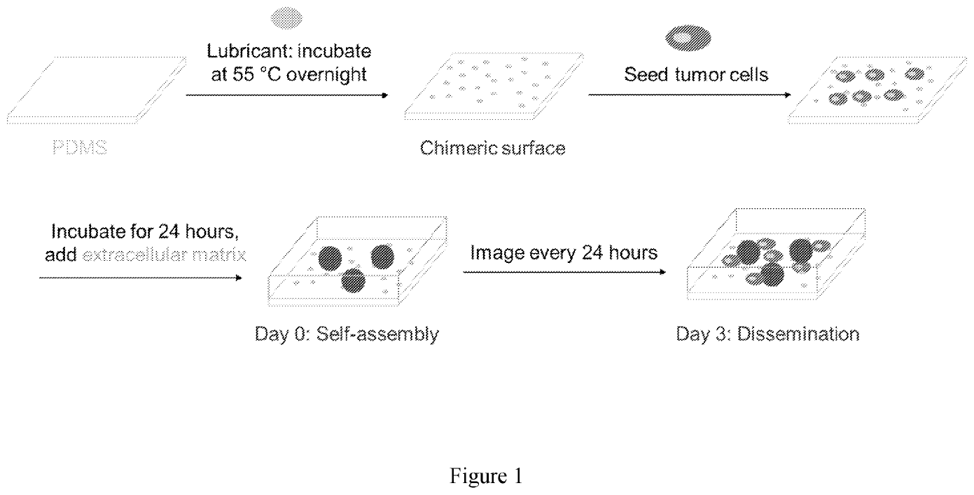

A chimeric surface was developed for tumor spheroids self-assembly and dissemination (FIG. 1). Human bladder cancer dissociated tumor cells were seeded on the chimeric surfaces constructed with Krytox filled PDMS. On day 0 (24 hours after the cell seeding), the tumor cells self-assembled and formed tumor organoids on the chimeric surface. These organoids were then disseminated, and aggressive cells were spread out in the following three days

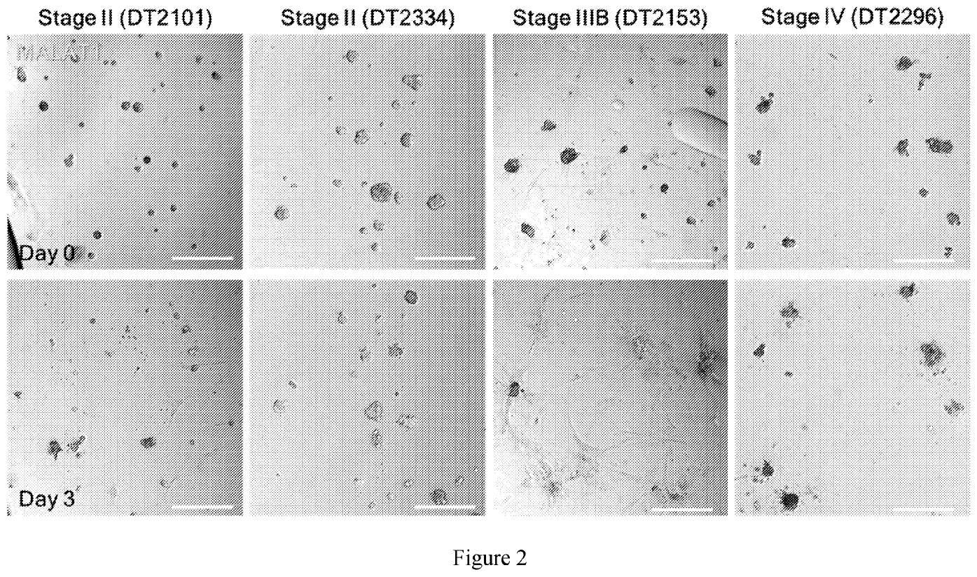

(FIG. 2). Interestingly, for two Stage II tumor cell samples (DT2101 and DT2334), a small number of aggressive cells were spread out on the chimeric surface, while a large number of aggressive cells were observed and spread out on Stage IIIB sample. In contrast, tumor cells from Stage IV sample did not disseminate from the bulk spheroids. There were many detached cells from the bulk organoids, indicating a distinct invasion mechanism compared to other samples.

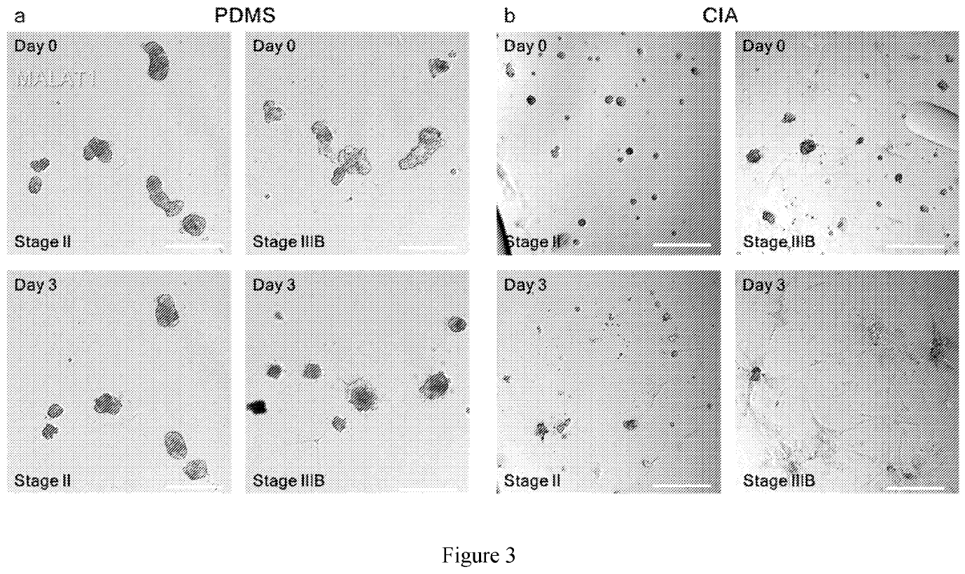

The mechanism of self-assembly and dissemination of the tumor organoids cultured on a chimeric surface was studied. The tumor cells were cultured on a PDMS surface and chimeric surface. On day 0, tumor organoids were self-assembled on both surfaces, while the tumor cells formed smaller organoids with more regular spheroid-shape on chimeric surface compared to PDMS. On day 3, tumor organoids formed on PDMS sprouted into the gel with clear leader-follower organization similar to the 3D invasion assay (FIG. 3). This indicated that the dissemination phenomenon is attributed to the addition of Krytox. The actin filaments of the tumor organoids were stained with siR-actin kit

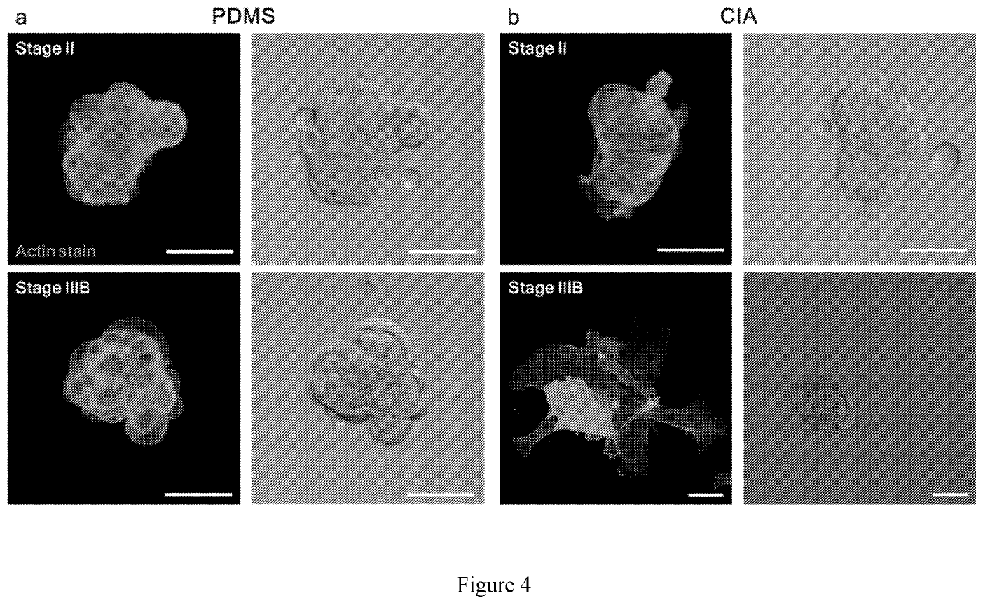

(FIG. 4). For Stage II sample cultured on PDMS and chimeric surface, no actin filaments were observed, indicating that the tumor cells from Stage II sample did not attach to neither of the surfaces. For Stage IIIB sample, the disseminated cells observed on chimeric surface show distinguishable actin filaments, indicating the tumor cells can spread out and attach to the chimeric surface but not PDMS.

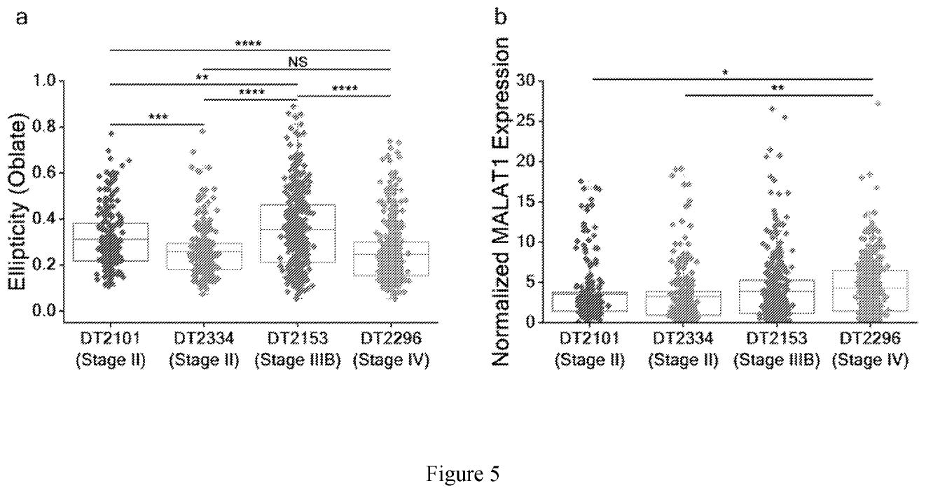

We quantified the ‘self-assembly to dissemination’ phenomena on CIA by measuring the ellipticity (oblate) and MALAT1 expression level of individual organoids or disseminated cells (FIG. 5). For a perfect sphere, the ellipticity is 0. The ellipticity increases with ‘flatter’ spheroids. When modeled as oblate spheroids, disseminated cells are flat and have a larger ellipticity. The results showed that Stage IIIB exhibited significantly higher ellipticity compared to Stage II samples. Stage IV sample invaded with a distinct mechanism and did not disseminate on the chimeric surface. Of note, the MALAT1 expression level in Stage IV samples is significantly higher than Stage II samples. We observed that MALAT1 expression level increases with higher cancer stage, although the statistical analysis does not show a significant correlation due to the deviation of large number of low MALAT1 expression cells.

Combining the cell dissemination (phenotype) and MALAT1 expression (genotype) of tumor samples, CIA provided more invasion features that cannot be studied with single assay.

### Chimeric Surface Characterization.

To study the ‘self-assembly to dissemination’ phenomena on CIA, several surface characterization methods were applied to study molecule component and distribution of chimeric surface.

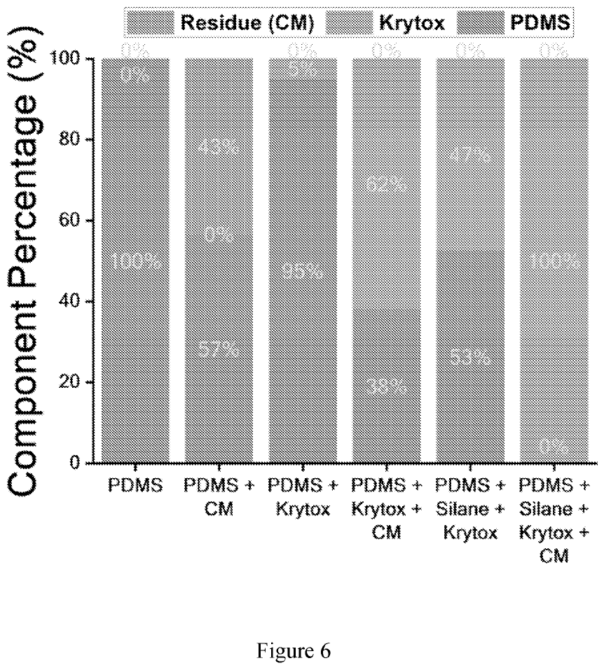

The goal of the surface characterization was to reveal the molecular structures on the surface. During the ‘self-assembly to dissemination’ phenomena of tumor spheroids, cancer cells interact with the surface. The hypothesis is the unique molecule structure or distribution leads to the phenomena. X-ray photoelectron spectroscopy (XPS) was used to study the molecule components. XPS analyzes the element component on the top 5 nm of the surface. The PDMS surface was treated with silane for a strong binding of Krytox as a positive control. By analyzing the elements on the surface, the molecule percentages of PDMS, Krytox, and protein can be calculated (FIG. 6). To mimic the culture condition, cell culture media was applied on the surface and measured the protein as the residue of the culture media. XPS detected 5% of Krytox on the chimeric surface, while the positive control surface retained 47% of Krytox. Interestingly, when incubated with cell culture media, the retention of Krytox on the chimeric surface increased to 62%, indicating the cell culture media may interact with Krytox and improve the binding of Krytox on the chimeric surface. 38% of the CIA surface is detected as PDMS, which explains the ‘self-assembly’ of tumor spheroids.

However, as the protein from the cell culture media was not detected by the XPS on the chimeric surface; it is hypothesized that the protein bonded with PDMS and was covered by Krytox.

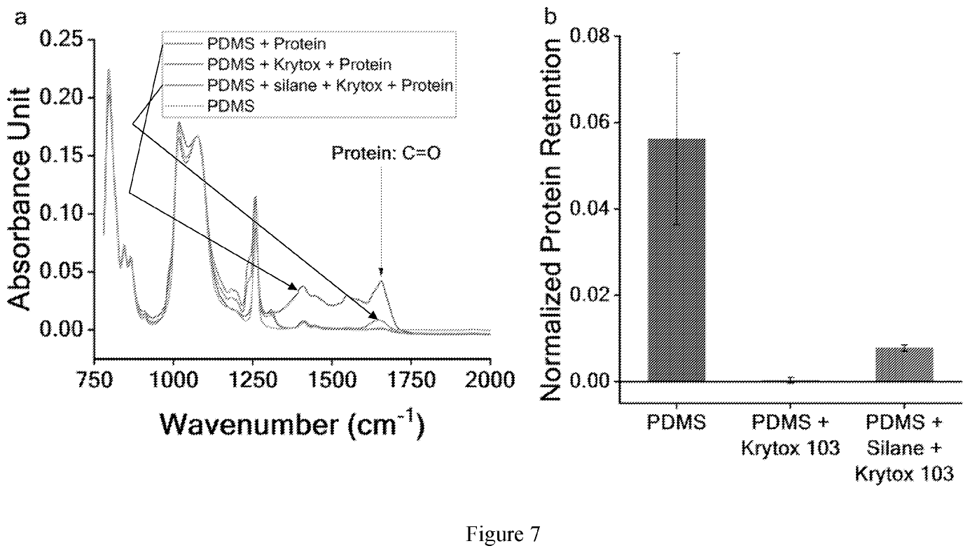

The chimeric surface was also characterized with Fourier-transform infrared spectroscopy (FTIR) that can penetrate 500 nm into the sample to detect the retention of protein (FIG. 7). Consistent with the XPS results, PDMS surface retained more protein compared to chimeric surface. Of note, the deviation of protein retention on PDMS surface was larger than the chimeric surface (FIG. 7b), indicating that the protein interacted with Krytox but not PDMS on the chimeric surface. It is speculated that the retention of protein and the protein-Krytox complex on CIA induce the ‘dissemination’ of tumor spheroids on CIA.

### Transurethral Resection of Bladder Tumor (TURBT) Analysis on CIA.

Finally, to validate the translational potential of CIA in bladder cancer prognosis, dissociated tumor cells from TURBT were cultured on CIA. The tumors obtained from the surgery were digested and the dissociated tumor cells were transfected with MALAT1 nanobiosensor and seeded on chimeric surface.

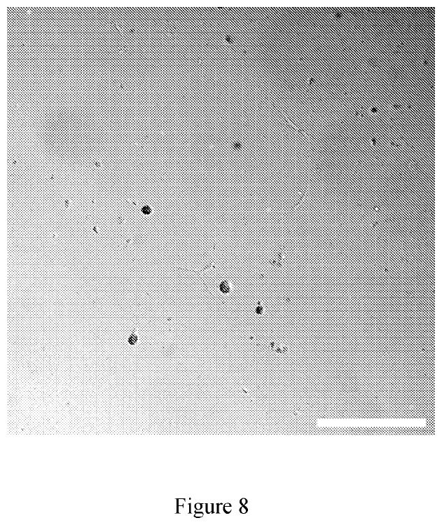

As shown in FIG. 8, the primary dissociated tumor cells self-assembled on the chimeric surface and disseminated cells were observed on day 3. Of note, commercially available 3D invasion assays failed to reconstruct tumor organoids because the assay is sensitive to cell viability. The CIA successfully combined the detection of tumor cell morphology and MALAT1 expression, suggesting promising potential for bladder cancer prognosis.

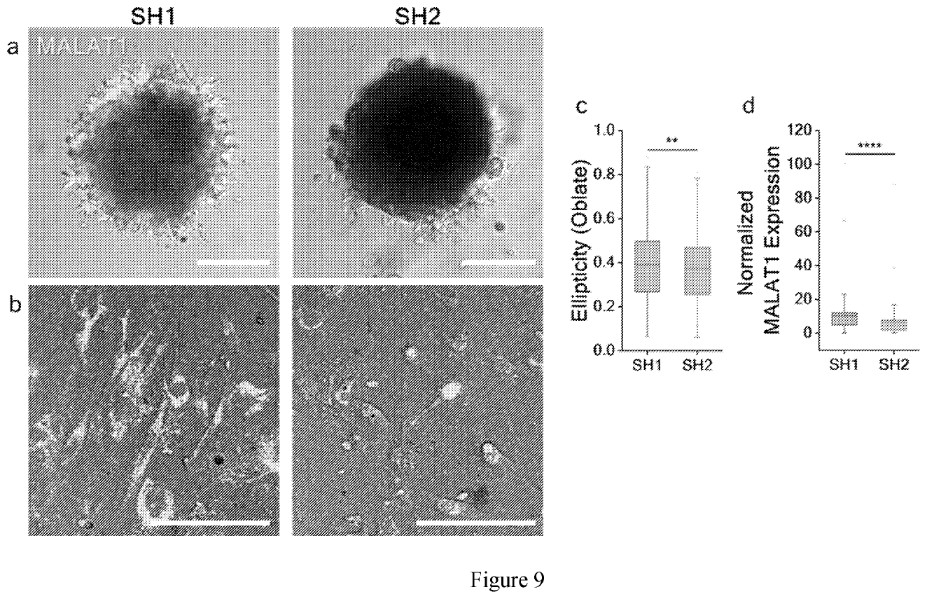

CIA prediction was further validated with 3D invasion assay by sub-cultured TURBT cells. The TURBT cells were expanded under the normal cell culture condition and were used to form tumor organoids with 3D invasion assay and CIA (FIG. 9). SHI exhibited more cell dissemination (FIG. 9b, c) and higher MALAT1 expression level (FIG. 9b, d) compared to SH1. Commercial 3D invasion assays showed a similar trend (FIG. 9a). The clinical histopathology indicated that SHI is high grade Stage II bladder cancer, and SH2 is high grade Stage I bladder cancer. The prediction from CIA correlated with the clinical prognosis.

As described herein, to demonstrate the translational potential of the MALAT1 biosensing and invasion assay in bladder cancer prognosis, TURBT cells were investigated. A novel invasion assay, named CIA, was developed to overcome the drawbacks of current 3D invasion assay, providing an intuitive readout that compatible with TURBT. When cultured on the chimeric surface, invasive tumor cells (Stage IIIB) first

self-assembled to form tumor organoids and then disseminated after invading for three days. This ‘self-assembly to dissemination’ phenomenon is less significant for less invasive tumor cells (Stage II), providing a promising assay for bladder cancer prognosis. Nevertheless, similar to the commercial 3D invasion assay, the chimeric surface cannot differentiate the Stage IV sample due to a distinct invasion mechanism, the chimeric surface requires the combination of MALAT1 biosensing for an accurate prognosis.

Surface characterization methods, XPS and FTIR, were conducted to understand the mechanism of the ‘self-assembly to dissemination’ phenomenon on the chimeric surface. Specifically, the distribution of Krytox in the surface and the interactions between the chimeric surface and the cell culture media need unveiling. The XPS surface characterization revealed that the addition of cell culture media increases the surface distribution density of Krytox. The FTIR results suggested that the protein in the cell culture media may interact with Krytox but not PDMS. These results indicated the possibility that the tumor cell self-assembly is caused by PDMS surface, and the dissemination is a result of interaction between aggressive cells and protein-Krytox complex. Further surface characterization methods with molecular level resolution are required to confirm this hypothesis.

Examining the dissociated tumor cells from fresh TURST tissues on CIA assay to provide bladder cancer prognosis is the ultimate goal. Interestingly, the SH2 primary tumor cells formed ‘self-assembly to dissemination’ phenomenon on CIA, showing a promising potential for applying CIA in clinical prognosis. Tumor organoids formation from sub-cultured TURBT samples showed good correlation between the commercial 3D invasion assay and CIA, supporting the bladder cancer prognosis potential of CIA.

Compared to the traditional 3D organoid analysis assay, tumor cells can ‘self-assemble’ on the CIA surface, allowing the high throughput analysis of tumor organoids while the embedded invasion assay can only form one spheroid per well. The property of ‘dissemination’ phenomenon provides intuitive observation of the invasiveness of the patient sample. The new CIA assay combines the phenotypic analysis of the tumor organoid morphology and the genotypic analysis of the RNA biosensor, providing comprehensive evidence for bladder cancer prognosis.

Although the present disclosure has been described with respect to one or more particular embodiments and/or examples, it will be understood that other embodiments and/or examples of the present disclosure may be made without departing from the scope of the present disclosure.

1. A thin film comprising one or more siloxanes and one or more lubricants, wherein at least a portion of the one or more lubricants is contacting a surface of the one or more siloxanes.

2. The thin film according to claim 1, wherein the one or more siloxanes are chosen from polydimethylsiloxanes (PDMS), polymethylhydrosiloxanes (PMHS), octamethylcyclotetrasiloxanes, and any combination thereof.

3. The thin film according to claim 2, wherein the one or more siloxanes are PDMS.

4. The thin film according to claim 2, wherein the PDMS comprises an elastomer component and a cross-linker component and cross-linker component has a concentration of 3.3 to 33% relative to the total percentage of elastomer and cross-linker.

5. The thin film according to claim 2, wherein the PDMS has ratio of 10:1 elastomer component to cross-linker component.

6. The thin film according to claim 1, wherein the one or more lubricants are perfluoroalkyl ethers (PFAEs).

7. The thin film according to claim 1, wherein the one or more lubricants have the following chemical structure: 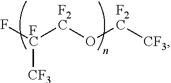
wherein n is 10 to 60.

8. The thin film according to claim 1, wherein the one or more lubricants have a viscosity of 7.8 cSt to 450 cSt.

9. The thin film according to claim 1, wherein the pores have a longest linear dimension of 0.2 nm to 20 μm.

10. The thin film according to claim 1, wherein the thin film has a thickness of 2 μm to 2 cm.

11. The thin film according to claim 1, wherein the thin film has a Young's modulus of 20 to 2500 kPa.

12. An article of manufacture having one or more surfaces, wherein the thin film according to claim 1 is disposed on at least a portion of at least one surface of the article of manufacture.

13. The article of manufacture according to claim 12, wherein the article of manufacture is a cell culture device.

14. The article of manufacture according to claim 13, wherein the cell culture device is a cell culture plate, a multi-well plate, a test tube, a flask, or a bioreactor.

15. A method of producing an organoid, comprising contacting one or more cells with a thin film according to claim 1.

16. The method according to claim 15, wherein the thin film is disposed on a surface of an article of manufacture.

17. The method according to claim 16, wherein the article of manufacture is a cell culture device.

18. The method according to claim 17, wherein the cell culture device is a cell culture plate, a multi-well plate, a test tube, a flask, or a bioreactor.

19. The method according to claim 15, wherein the one or more cells are mammalian cells.

20. The method according to claim 19, wherein the mammalian cells are cancer cells.

21. The method according to claim 20, wherein the cancer cells are bladder cancer cells.

22. A method for determining if a subject has cancer, comprising culturing one or more cells isolated from the subject in the presence of a thin film of claim 1, wherein the formation of an organoid in contact with the thin film is indicative of the subject having cancer.

23. The method according to claim 22, wherein the cancer is bladder cancer.
